(19) United States
(12) Patent Application Publication
(10) Pub. No.: US 2011/0001829 A1
(43) Pub. Date: Jan. 6, 2011

(54) MULTIFUNCTIONAL X-RAY READER

(75) Inventor: Jung-Chang LAI, Taoyuan County (TW)

Correspondence Address: BRIAN M. MCINNIS, 12th Floor, Ruttonjee House, 11 Duddell Street, Hong Kong, HK

(73) Assignee: Jung-Chang LAI, Taoyuan County (TW)

(21) Appl. No.: 12/651,380

(22) Filed: Dec. 31, 2009

(30) Foreign Application Priority Data
Jul. 2, 2009 (TW) 98122443

(51) Int. Cl. H04N 5/32

(52) U.S. Cl. 348/162; 348/E05.086

(57) ABSTRACT

A multifunctional X-ray reader includes an enclosure assembled from a front and a rear enclosure to define a receiving chamber for receiving at least one circuit board therein; a liquid crystal display (LCD) screen provided on the front enclosure and electrically connected to the circuit board; at least one push-button group arranged on the enclosure and electrically connected to the circuit board; an X-ray film tray outward extendably received in the enclosure for holding an X-ray film thereon; and an image capture unit located in the enclosure to face toward the X-ray film tray and electrically connect to the circuit board. The image capture unit captures an image data from the X-ray film, and the captured image data is displayed on the LCD display screen and simultaneously stored in a memory card held in a memory card holder provided on the enclosure and electrically connected to the circuit board.

### RELATED APPLICATIONS

This application claims priority to Taiwan Application Serial Number 98122443, filed Jul. 2, 2009, which is herein incorporated by reference.

### FIELD OF THE INVENTION

The present invention relates to an X-ray reader, and more particularly to a multifunctional X-ray reader applied in intra-oral radiography.

### BACKGROUND OF THE INVENTION

In dental treatment, it is often necessary to make a radiograph of the patient's oral cavity to get an X-ray image showing the affected parts of teeth and gingival cartilage tissues. Then, the X-ray image is studied to determine the conditions of the affected parts.

Conventionally, the X-ray image can be presented in two manners. In the first manner, an X-ray viewer box is used to show the X-ray image. FIG. 1 shows a conventional X-ray viewer box 110 being mounted to a dental chair 200. The X-ray viewer box 110 is provided with a backlight source. When an X-ray film is attached to the X-ray viewer box 110, the X-ray film is illuminated by the backlight source to show the X-ray image thereon. In the process of dental treatment, both the dentist and the patient lying on the clinic chair can know and discuss the affected part by viewing the X-ray viewer box 110 to facilitate the subsequent treatment procedures.

However, the conventional X-ray viewer box 110 can only be used to present the x-ray image of the affected part without the function of converting the image into digital data for writing into and reading from a storage device or transmitting between two different devices, and establishing an electronic case history for the patient.

In the second manner, an X-ray reader connected to a computer and a display is used to show the X-ray image. FIG. 2 shows a conventional X-ray reader 120 being connected to a computer 130 and a display 140 for use with a dental chair 200. The X-ray reader 120 is able to convert the X-ray image into digital image data and transmit the same to the computer 130 and the display 140 via cables for displaying on the display 140. The display 140 is mounted on the clinic chair 200, allowing both the dentist and the patient to observe and examine the X-ray image of the affected part shown on the display 140.

In the second manner, the X-ray reader 120 must be used with the computer 130 and the display 140 to show the X-ray image of the affected part and store the image data on the computer 130. As a whole, such arrangement is complicated. However, the conventional X-ray reader 120 can only read the X-ray image without the image display and data storage functions. Further, the conventional X-ray reader 120 could not be used with an intra-oral dental camera or take the place of the existing X-ray viewer box 110.

### SUMMARY OF THE INVENTION

A primary object of the present invention is to provide a multifunctional X-ray reader, which also provides image display and data storage functions and can be used with an intra-oral dental camera.

To achieve the above and other objects, the multifunctional X-ray reader according to the present invention includes an enclosure having a front enclosure and a rear enclosure, which are fastened to each other to define a receiving chamber in the enclosure, at least one circuit board being received in the receiving chamber, and a liquid crystal display (LCD) screen being provided on a front face of the front enclosure and electrically connected to the circuit board; at least one push-button group arranged on the enclosure and electrically connected to the circuit board; an X-ray film tray outward extendably received in the enclosure for holding an X-ray film thereon; and an image capture unit including a lens holder and a lens held in the lens holder. The image capture unit is located in the enclosure to face toward the X-ray film tray and electrically connect to the circuit board for capturing an image data from the X-ray film and outputting the captured image data to the LCD screen. Accordingly, when the image capture unit captures the image data from the X-ray film, the captured image is simultaneously displayed on the LCD screen.

According to an embodiment of the multifunctional X-ray reader of the present invention, a memory card holder is provided on the enclosure and electrically connected to the circuit board. A memory card can be inserted into the memory card holder for storing the image data captured by the image capture unit from the X-ray film.

According to an embodiment of the present invention, there are two push-button groups arranged on the enclosure, including a first push-button group and a second push-button group. The first and the second push-button group are respectively arranged on the front and the rear enclosure for a user to select different operations for controlling the X-ray reader.

According to an embodiment of the present invention, a mount is provided in the receiving chamber of the enclosure. The mount has a first end and an opposing second end. The X-ray film tray and the image capture unit are respectively mounted to the first and the second end of the mount.

According to another embodiment of the present invention, the mount is assembled from a first mount section and an opposing second mount section. The first and the second end of the mount are located at the first and the second mount section, respectively.

According to an embodiment of the present invention, a light guide plate is provided to one side of the X-ray film tray facing away from the image capture unit and mounted on the first mount section for guiding light.

According to an embodiment of the present invention, a connector unit is provided on the enclosure to electrically connect to the circuit board. The connector unit includes a fast connector and a plurality of input/output connectors for connecting the X-ray reader to other external instruments.

According to an embodiment of the present invention, the multifunctional X-ray reader further includes a pivot support unit having an insertion slot and a pivot shaft, and the enclosure has a shaft holder corresponding to the pivot shaft. The pivot shaft of the pivot support unit is pivotally turnably received in the shaft holder provided on the enclosure. The pivot support unit supports the X-ray reader thereon, and the enclosure can be turned about the pivot shaft of the pivot support unit to adjust an inclination angle of the LCD screen located on the enclosure.

According to an embodiment of the present invention, the pivot support unit includes a first cover plate and a second cover plate, which are closed and connected to each other to define the insertion slot and the pivot shaft therebetween.

According to an embodiment of the present invention, a connecting bracket can be inserted in the insertion slot of the pivot support unit. The connecting bracket has a first insertion section and an opposing second insertion section. The first insertion section of the connecting bracket is inserted in the insertion slot of the pivot support unit, while the second insertion section of the connecting bracket is inserted in an insertion hole on a dental chair, so that the X-ray reader is fixed on the dental chair.

Alternatively, a supporting stand can be inserted in the insertion slot of the pivot support unit. The supporting stand has a base portion and a connecting bracket portion connected to the base portion. The connecting bracket portion of the supporting stand is inserted in the insertion slot of the pivot support unit, while the base portion has a large supporting area for stably positioning the X-ray reader on a platform.

According to an embodiment of the present invention, an intra-oral dental camera holder is provided to one side of the enclosure for holding an intra-oral dental camera therein, so that the X-ray reader can be used with the intra-oral dental camera.

According to an embodiment of the present invention, the intra-oral dental camera holder includes an arm portion and a cup portion connected to the arm portion. The arm portion is connected to the enclosure, and the cup portion is used to hold the intra-oral dental camera therein.

The intra-oral dental camera is equipped with a display screen. An x-ray image of the affected part captured using the intra-oral dental camera can be synchronously displayed on the display screen. Therefore, an operator can directly see the captured image on the display screen in the process of treatment.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 shows a conventional X-ray viewer box being mounted to a dental chair;

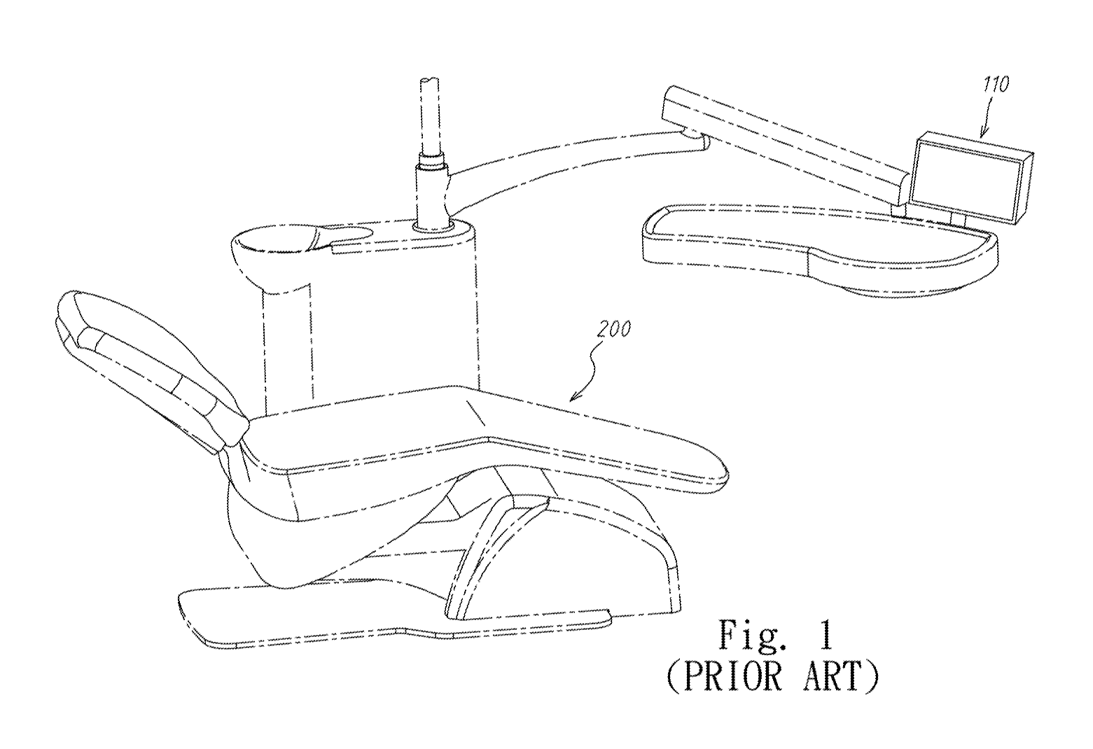

FIG. 2 shows a conventional X-ray reader being connected to a computer and a display for use with a dental chair;

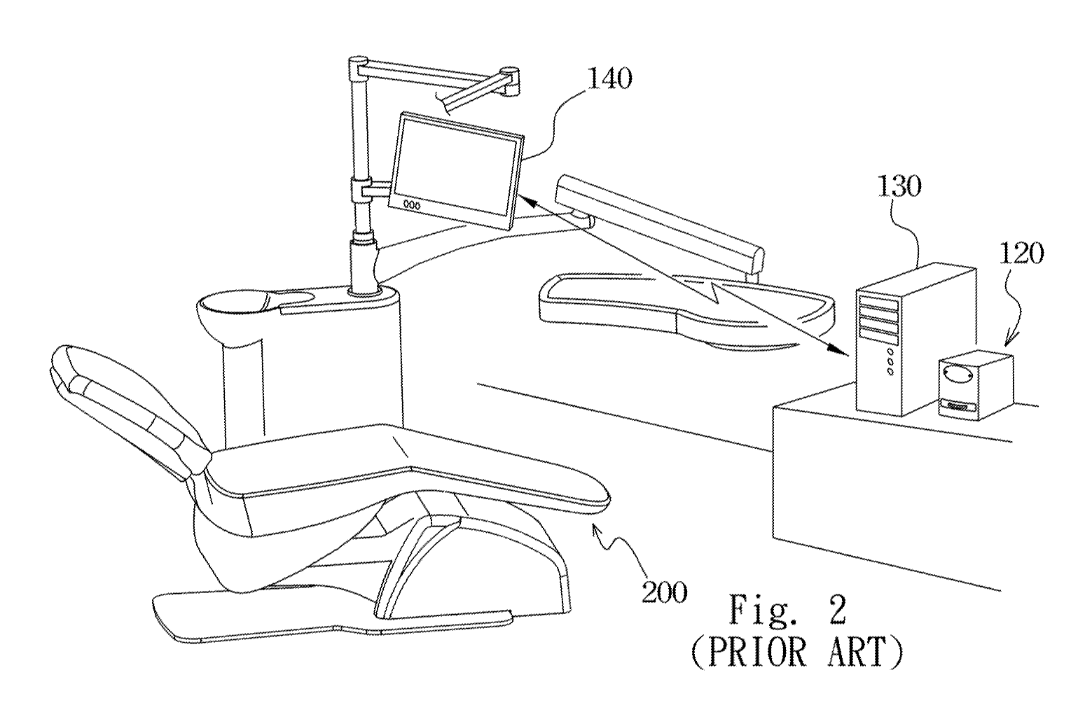

FIG. 3 is an assembled front perspective view of a multifunctional X-ray reader according to an embodiment of the present invention;

FIG. 4 is an exploded perspective view of FIG. 3, in which a connecting bracket, a supporting stand, and an intra-oral dental camera holder are further shown;

FIG. 5 is a sectioned side view of FIG. 3;

FIG. 6 shows the Multifunctional X-ray reader of FIG. 3 with a connecting bracket connected thereto;

FIG. 7 is a rear view of the multifunctional X-ray reader FIG. 6 with an intra-oral dental camera holder provided thereto;

FIG. 8 is a sectioned side view of FIG. 6;

FIG. 9 shows the multifunctional X-ray reader of the present invention as shown in FIG. 7 being mounted to a dental chair for use; and

FIG. 10 shows the multifunctional X-ray reader of FIG. 3 with a supporting stand and an intra-oral dental camera holder connected thereto.

### DETAILED DESCRIPTION OF THE PREFERRED EMBODIMENTS

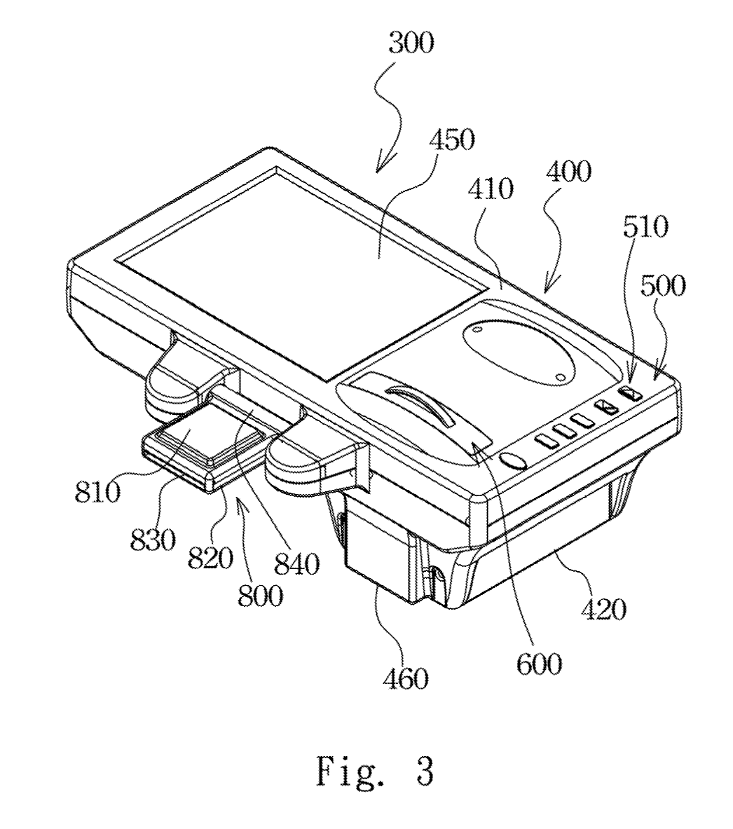

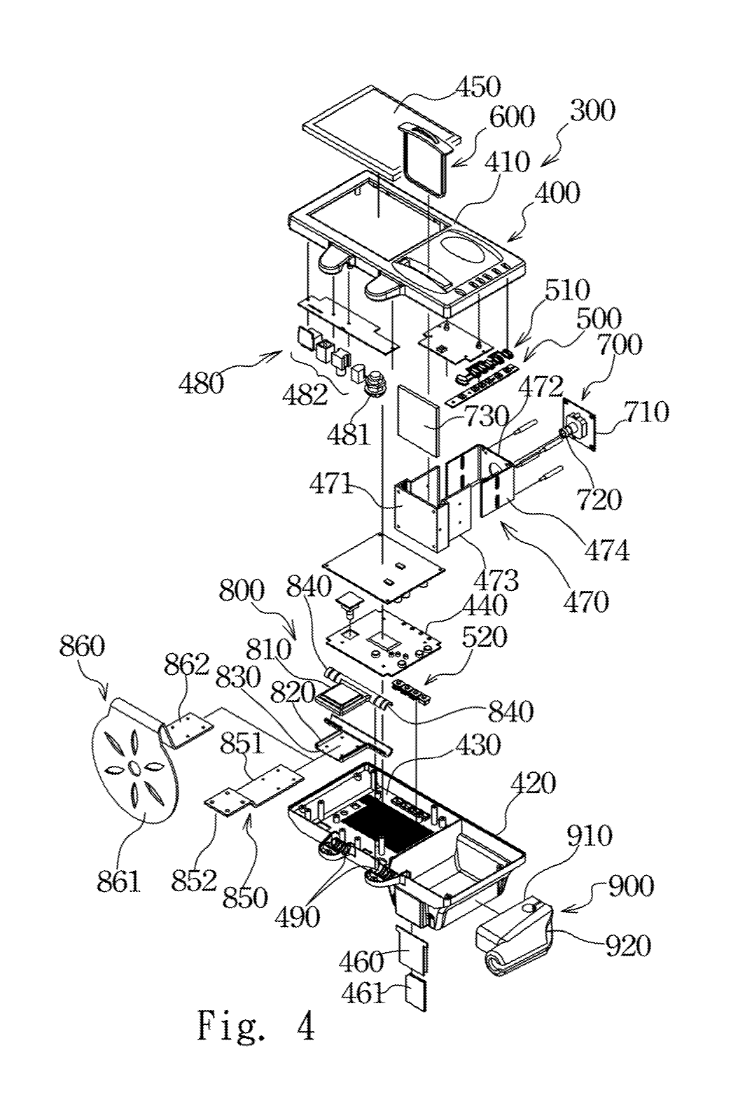

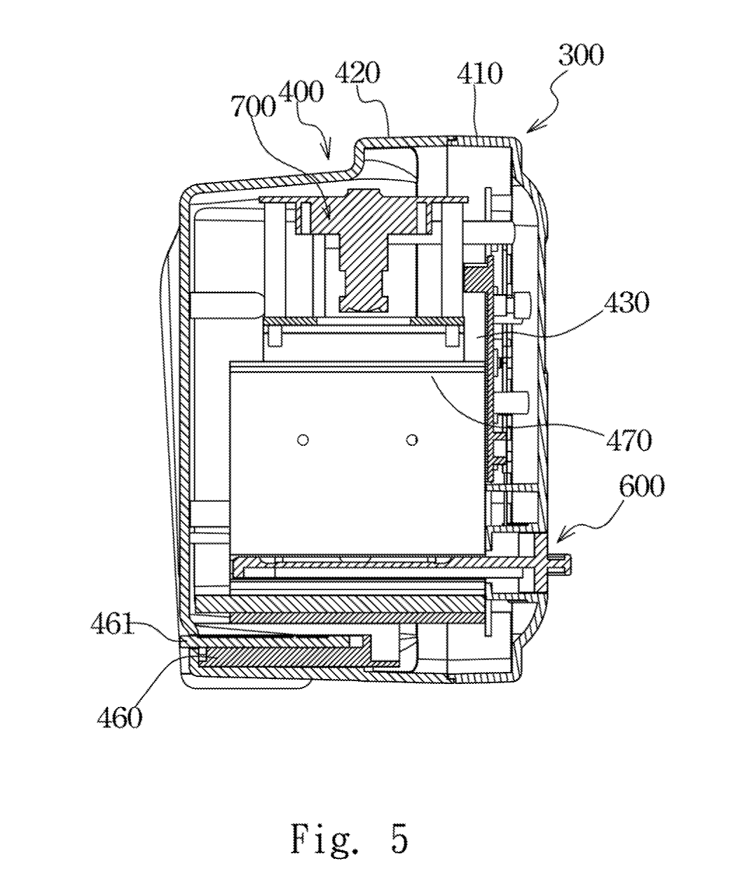

Please refer to FIGS. 3 to 5, in which FIG. 3 is an assembled front perspective view of a multifunctional X-ray reader 300 according to an embodiment of the present invention, and FIGS. 4 and 5 are exploded and sectioned side views, respectively, of FIG. 3. As shown, the multifunctional X-ray reader 300 includes an enclosure 400, at least one push-button group 500 (a first push-button group 510 and a second push-button group 520 are provided in the illustrated embodiment), an X-ray film tray 600, and an image capture unit 700.

The enclosure 400 includes a front enclosure 410 and a rear enclosure 420, which are fastened to each other to define a receiving chamber 430 in the enclosure 400. At least one circuit board 440 having a driving circuit, a conversion circuit, and many other different circuits provided thereon is arranged in the receiving chamber 430. A liquid crystal display (LCD) screen 450 is provided on a front face of the front enclosure 410 and electrically connected to the circuit board 440.

The first and second push-button groups 510, 520 are respectively arranged on the front and rear enclosures 410, 420 and electrically connected to the circuit board 440. Each of the first and second push-button groups 510, 520 has a plurality of push buttons, such as a Page Up button, a Page Down button, a Pause/Capture button, etc., for a user to select different operations for controlling the X-ray reader 300.

The X-ray film tray 600 is outward extendably received in the enclosure 400 for holding an X-ray film (not shown) thereon. Through radiography, an x-ray image of an affected part in a patient's oral cavity is kept on the X-ray film for viewing and examining by a dentist.

The image capture unit 700 includes a lens holder 710 and a lens 720 held in the lens holder 710. The image capture unit 700 is located in the enclosure 400 facing toward the X-ray film tray 600 and electrically connected to the circuit board 440. A light guide plate 730 is provided on one side of the X-ray film tray 600 facing away from the image capture unit 700 for guiding light. The image capture unit 700 captures the x-ray image on the X-ray film. The captured x-ray image is converted by the circuit board 440 into digital image data, which is then output to and directly shown on the LCD screen 450.

A mount 470 is provided in the receiving chamber 430 of the enclosure 400. The mount 470 has a first end 471 and an opposing second end 472. The X-ray film tray 600 and the image capture unit 700 are respectively mounted to the first and the second end 471, 472 of the mount 470. The mount 470 can be an integrally formed structure. Alternatively, the mount 470 can be assembled from a first mount section 473 and a second mount section 474. In this case, the first and the second end 471, 472 of the mount 470 are located at the first and the second mount sections 473, 474, respectively. The light guide plate 730 is also arranged on the first mount section 473.

Further, a memory card holder 460 is provided on the enclosure 400 and electrically connected to the circuit board 440. A memory card 461 can be inserted into the memory card holder 460. The image data captured by the image capture unit 700 from the X-ray film held in the X-ray film tray 600 can also be simultaneously stored in the memory card 461. Therefore, data of the captured image can be written into or read from the memory card 461 and can be carried to different locations for transmitting. The captured x-ray image can also be used to establish an electronic case history for the patient.

Moreover, a connector unit 480 is provided on the enclosure 400 to electrically connect to the circuit board 440. The connector unit 480 includes a fast connector 481 for quickly connecting the X-ray reader 300 to an external display device in a wired or a wireless manner. The connector unit 480 also includes a plurality of input/output connectors 482, such as a DC (direct current) input connector, an AV (audio/visual) output connector, a USB (universal serial bus) output connector, a VGA (video graphics array) output connector, etc., via which the X-ray reader 300 can be connected to other relevant instruments.

The multifunctional X-ray reader 300 of the present invention further includes a pivot support unit 800 for supporting the X-ray reader 300 on a desired position. The pivot support unit 800 includes a first cover plate 810 and a second cover plate 820, which are closed and connected to each other to define an insertion slot 830 and a pivot shaft 840. The enclosure 400 is provided with a shaft holder 490 corresponding to the pivot shaft 840, such that the pivot shaft 840 of the pivot support unit 800 can be pivotally turnably received in the shaft holder 490 on the enclosure 400. With these arrangements, the enclosure 400 can be turned about the pivot shaft 840 relative to the first and second cover plates 810, 820 of the pivot support unit 800 to adjust an inclination angle of the LCD screen 450 located on the enclosure 400.

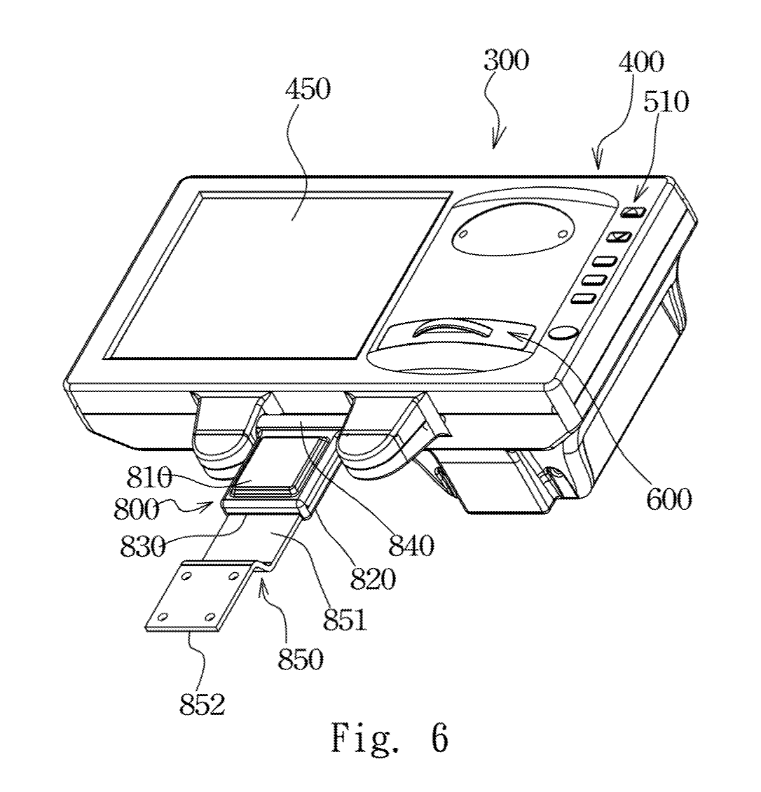

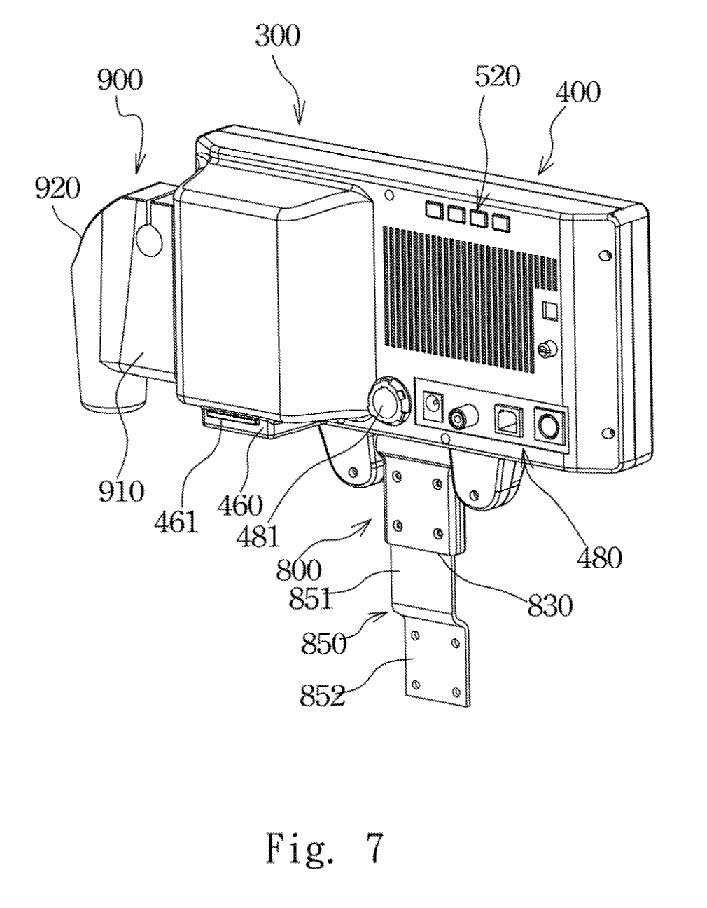

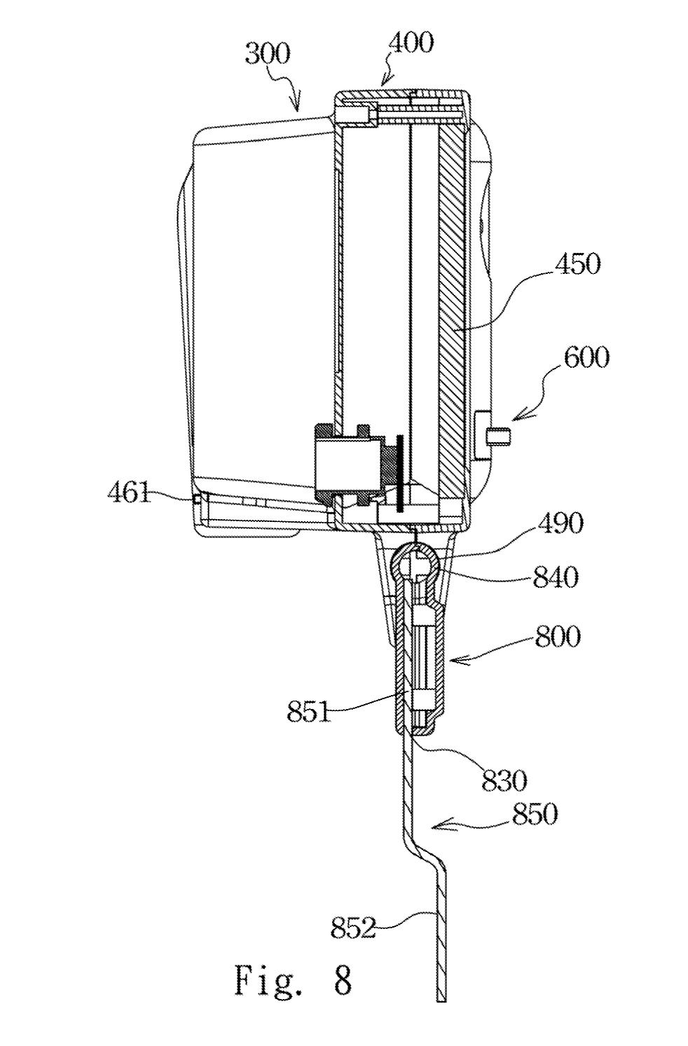

The enclosure 400 and the pivot support unit 800 of the X-ray reader 300 can be supported on a fixed device via a corresponding member. For example, as shown in FIGS. 6 to 9, the X-ray reader 300 is fixedly connected to a dental chair 200 via a connecting bracket 850. More specifically, the connecting bracket 850 has a first insertion section 851 and an opposing second insertion section 852. The first and the second insertion section 851, 852 are respectively inserted in the insertion slot 830 of the pivot support unit 800 and an insertion hole on the dental chair 200. Via the connecting bracket 850, the X-ray reader 300 can be fixed on the dental chair 200. In the process of treatment, both the dentist and the patient lying on the dental chair 200 can watch the LCD screen 450 on the multifunctional X-ray reader 300 to know the exact condition of the affected part in the patient's oral cavity.

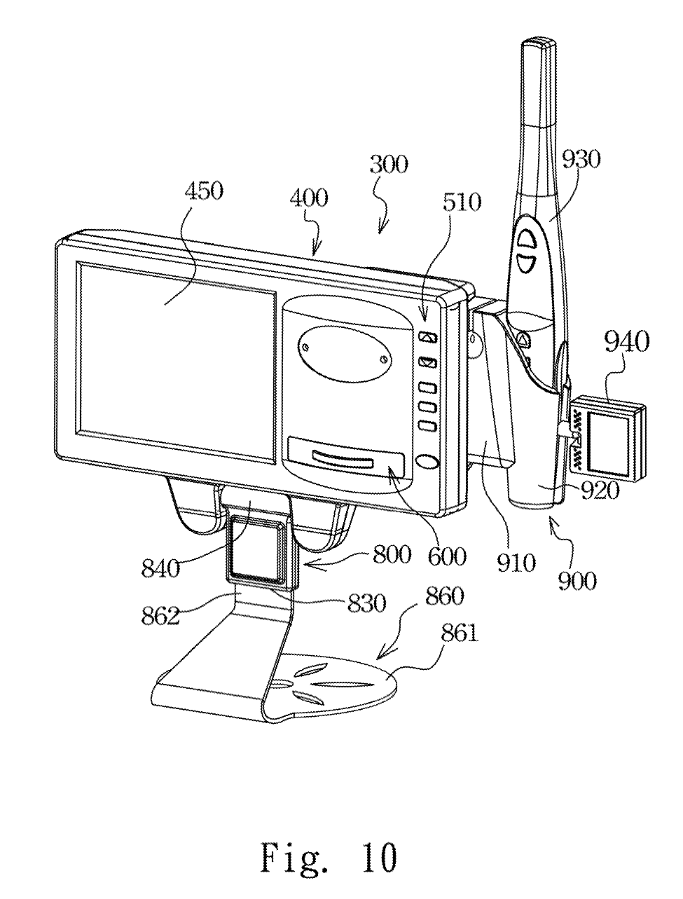

FIG. 10 shows another manner of supporting the multifunctional X-ray reader 300 on a fixed device. As shown, the multifunctional X-ray reader 300 can be supported on a platform via a supporting stand 860 for use in the case of, for example, a home-visit treatment where a dental chair is not available. More specifically, the supporting stand 860 has a base portion 861 and a connecting bracket portion 862 connected to the base portion 861. The connecting bracket portion 862 is inserted in the insertion slot 830 of the pivot support unit 800, and the base portion 861 has a large supporting area for stably positioning the X-ray reader 300 on the platform. In the process of treatment, both the dentist and the patient can watch the LCD screen 450 on the multifunctional X-ray reader 300 to know the exact condition of the affected part in the patient's oral cavity.

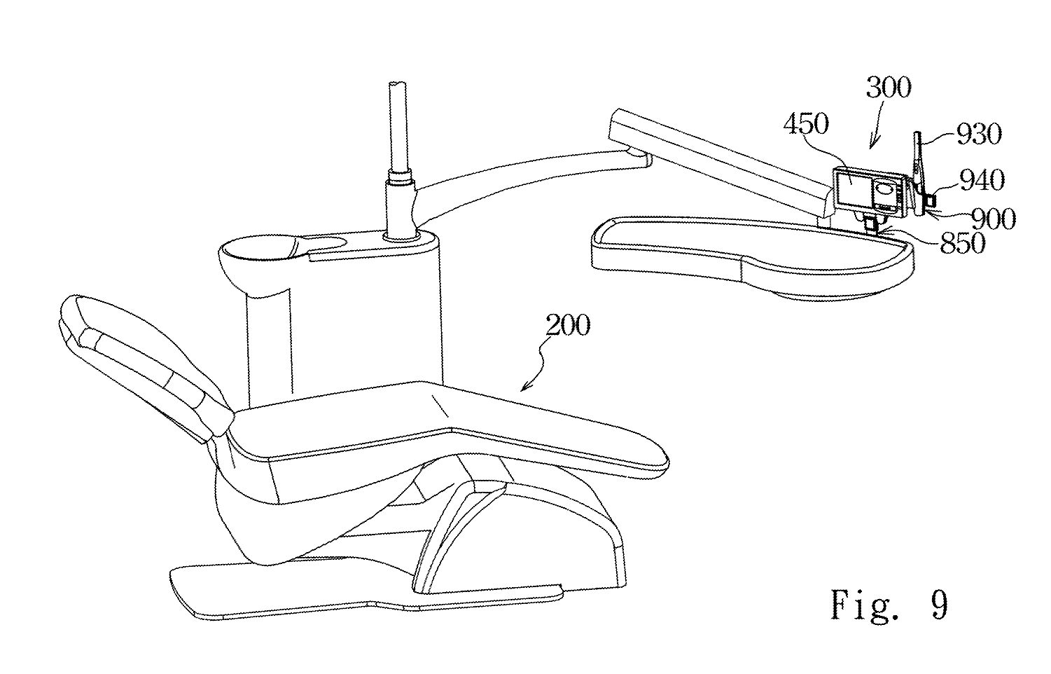

Please refer to FIGS. 9 and 10. The multifunctional X-ray reader 300 of the present invention can further include an intra-oral dental camera holder 900 mounted to one side of the enclosure 400. The intra-oral dental camera holder 900 includes an arm portion 910 connected to the enclosure 400 and a cup portion 920 connected to the arm portion 910 for holding an intra-oral dental camera 930 therein. With these arrangements, the intra-oral dental camera 930 can be directly positioned on and connected to the X-ray reader 300, and image data of the patient's affected part captured with the intra-oral dental camera 930 can be stored in the memory card 461. When the memory card 461 having the image data stored therein is inserted in the memory card holder 460, the image data of the patient's affected part can be directly shown on the LCD screen 450. In addition, the intra-oral dental camera 930 itself is also equipped with a display screen 940, the image of the affected part captured using the intra-oral dental camera 930 can be synchronously displayed on the display screen 940. Therefore, an operator, such as the dentist, can directly see the captured image on the display screen 940 in the process of treatment.

In conclusion, the multifunctional X-ray reader 300 of the present invention also provides the image display and data storage functions and can be used with an intra-oral dental camera, making it convenient and practical for use in a wide range of applications.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A multifunctional X-ray reader, comprising:
an enclosure including a front enclosure and a rear enclosure, which are fastened to each other to define a receiving chamber in the enclosure; at least one circuit board being arranged in the receiving chamber, and a liquid crystal display (LCD) screen being provided on a front face of the front enclosure and electrically connected to the circuit board;
at least one push-button group being arranged on the enclosure and electrically connected to the circuit board;
an X-ray film tray being outward extendably received in the enclosure for holding an X-ray film thereon; and
an image capture unit including a lens holder and a lens held in the lens holder, and being located in the enclosure to face toward the X-ray film tray and electrically connect to the circuit board for capturing image data from the X-ray film and outputting the captured image data to the LCD screen.

2. The multifunctional X-ray reader as claimed in claim 1, wherein the enclosure is provided with a memory card holder electrically connected to the circuit board for a memory card to insert therein; and the image data captured by the image capture unit from the X-ray film being simultaneously stored in the memory card.

3. The multifunctional X-ray reader as claimed in claim 2, wherein two push-button groups, including a first push-button group and a second push-button group, are respectively arranged on the front and the rear enclosures.

4. The multifunctional X-ray reader as claimed in claim 2, wherein a mount is provided in the receiving chamber of the enclosure; the mount having a first end and an opposing second end, and the X-ray film tray and the image capture unit being mounted to the first and the second end of the mount, respectively.

5. The multifunctional X-ray reader as claimed in claim 4, wherein the mount is assembled from a first mount section and an opposing second mount section, and the first and the second end being located on the first and the second mount section, respectively.

6. The multifunctional X-ray reader as claimed in claim 5, wherein a light guide plate is arranged on the first mount section to locate at one side of the X-ray film tray facing away from the image capture unit.

7. The multifunctional X-ray reader as claimed in claim 2, wherein the enclosure is provided with a connector unit electrically connected to the circuit board, and the connector unit including a fast connector and a plurality of input/output connectors.

8. The multifunctional X-ray reader as claimed in claim 2, further comprising a pivot support unit having an insertion slot and a pivot shaft connected to each other, and the enclosure being provided with a shaft holder corresponding to the pivot shaft, whereby the pivot shaft of the pivot support unit is pivotally turnably received in the shaft holder of the enclosure.

9. The multifunctional X-ray reader as claimed in claim 8, wherein the pivot support unit includes a first cover plate and a second cover plate, which are closed and connected to each other to define the insertion slot and the pivot shaft therebetween.

10. The multifunctional X-ray reader as claimed in claim 8, wherein a connecting bracket is inserted in the insertion slot of the pivot support unit, the connecting bracket having a first insertion section and an opposing second insertion section, and the first insertion section of the connecting bracket being inserted in the insertion slot of the pivot support unit.

11. The multifunctional X-ray reader as claimed in claim 10, wherein the second insertion section of the connecting bracket is inserted in a hole provided on a dental chair.

12. The multifunctional X-ray reader as claimed in claim 8, wherein a supporting stand is inserted in the insertion slot of the pivot support unit, the supporting stand having a base portion and a connecting bracket portion connected to the base portion, the connecting bracket portion of the supporting stand being inserted in the insertion slot of the pivot support unit.

13. The multifunctional X-ray reader as claimed in claim 1, wherein an intra-oral dental camera holder is mounted to one side of the enclosure for holding an intra-oral dental camera therein.

14. The multifunctional X-ray reader as claimed in claim 13, wherein the intra-oral dental camera holder has an arm portion and a cup portion connected to the arm portion, the arm portion being connected to the enclosure, and the cup portion being used to hold the intra-oral dental camera therein.

15. The multifunctional X-ray reader as claimed in claim 13, wherein the intra-oral dental camera is equipped with a display screen.
